(19) United States
(12) Patent Application Publication
(10) Pub. No.: US 2024/0269445 A1
(43) Pub. Date: Aug. 15, 2024

(54) IMPLANT FOR THE SUSTAINED RELEASE OF AN ACTIVE INGREDIENT

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 18/435,281

(22) Filed: Feb. 7, 2024

(30) Foreign Application Priority Data
Feb. 15, 2023 (EP) 23156684.5

(51) Int. Cl. A61M 37/00

(52) U.S. Cl. CPC A61M 37/00

(57) ABSTRACT

An implant for the sustained subcutaneous release of an active ingredient, which implant comprises a housing, which is delimited on a first side by a septum, and an outlet on a second side for the release of an active ingredient, and a reservoir, which is arranged in a cavity of the housing and is designed to receive a liquid containing an active ingredient.

### CROSS-REFERENCE TO RELATED APPLICATIONS

This application claims priority pursuant to 35 U.S.C. 119(a) to European Application No. 23156684.5, filed Feb. 15, 2023, which application is incorporated herein by reference in its entirety.

### FIELD OF THE INVENTION

The present invention relates to the field of medical technology, in particular to implantable devices for the delivery of active ingredients with sustained release. The implants can be implanted subcutaneously in spatial proximity to joints, for example knee joints. Possible applications are, for example, the treatment of inflammatory joint diseases, such as rheumatoid arthritis.

### TECHNICAL BACKGROUND

Rheumatic diseases belong to the so-called autoimmune diseases in which the immune system acts on the body's own structures for reasons which are as yet not precisely known. The diseases rheumatoid arthritis and psoriatic arthritis belong to rheumatic disorders and are associated with joint inflammation (arthritides). This typically chronic joint inflammation can lead to the progressive destruction of the joints. The joints of humans are surrounded by a joint capsule (Capsula articulatis). It is composed of an outer connective tissue layer (Membrana fibrosa) and an inner layer of the so-called synovial membrane (Membrana synovialis). The synovial membrane is crucial for the inflammatory processes in rheumatoid arthritis and psoriatic arthritis, because misdirected immune cells migrate through said membrane, or because the proliferation of fibroblasts on the synovial membrane can cause a pannus to be formed—which is a cell clone of fibroblasts. Upon the pannus disintegrating and as a result of the migrated immune cells, inflammation mediators, such as interleukins, and α-TNF (α-tumor necrosis factor) are secreted. These inflammatory mediators control the release of proteases and other destructive enzymes from immune cells via an inflammation cascade, which cells then lead to enzymatic destruction of the cartilage and bone structures.

Implants for delivering active pharmaceutical ingredients in the region of the joints are already known, said implants being used for the local application of antibiotics. Examples thereof include documents GB 2 290 971 A, US 2010/0042213 A, WO 2017/178951 A1, WO 2007/084878 A1, and U.S. Pat. No. 6,245,111 B1. When using these implants, however, the joint has to be removed extensively, and the implants primarily serve to treat the prostheses with antibiotics. Such implants are not considered for the treatment of rheumatoid arthritis or psoriatic arthritis because the joint to be treated would have to be removed for therapy and, as a result, the subsequent treatment would be pointless. US 2007/0219471 A1 discloses an implant for applying active ingredients and maintaining a reduced pressure for accelerating wound healing in cavities and for mechanically stabilizing cavities that do not heal well.

WO 2010/088548 A1 discloses a tubular, refillable eye implant with which a pharmaceutical active ingredient can be delivered directly to the eyeball. For this purpose, the tubular implant is inserted directly into the eyeball. The implant is not provided for or suitable for implanting in a joint. Insertion into the joint would cause the joint itself to be attacked and damaged, and upon inserting the implant there would be a risk of infection and inflammation of the joint.

EP3978065 discloses a disk-shaped implant made of a flexible material having a cavity which can be filled with an active ingredient solution using a cannula, wherein the implant expands elastically. The restoring force of the implant causes the active ingredient solution to leak out of microbores on the underside of the implant.

U.S. Pat. No. 5,681,289 A and US 2018/0028320 A1 disclose implants in which a medical fluid for treating joints or for lubricating joints is supplied to the joints from a reservoir via tubes. The implants are relatively large and are hard to position. A short-term change in medical treatment is complicated because the supply lines to the planar applicators would have to be flushed first, which is not readily possible when in the implanted state. The implants are well suited for lubricating joints with a lubricant delivered via the implant, while a targeted release of active pharmaceutical ingredients with a high concentration is not possible using these implants because too large volumes are contained in the implant, the reservoir and the supply line.

US 2003/0139811 A1 discloses an active ingredient system for the local release of anti-inflammatory active ingredients in the interior of joint capsules. These are hollow screws with an opening in the screw head. These hollow screws can be filled with an active ingredient. Subsequently, the screws are introduced into bone tissue, which is located next to the cartilage tissue within the joint capsule. The active ingredient should then be released into the synovial fluid via the opening in the screw head. A critical aspect here is that the joint capsule has to be opened and that the screw remains in the interior of the joint capsule after release of the active ingredient. Opening the joint capsule is always subject to a significant risk of infection. There is no provision for filling the implanted screw with new active ingredient. Therefore, the treatment can also not be adapted to a change in the state of the patient treated.

### PREFERRED EMBODIMENTS

The object of the present invention is to solve one or more of the problems described above and further problems of the prior art. For example, the invention facilitates a sustained release of an active ingredient from an implant. Furthermore, the present invention provides an implant which, in the unchanged implanted state, can be filled with active ingredient using an injection cannula.

These objects are achieved by the methods and devices described herein, in particular those which are described in the claims.

Preferred embodiments of the invention are described below.

A first embodiment describes an implant for the sustained subcutaneous release of an active ingredient, which implant comprises a housing, which is delimited on a first side by a septum and comprises an outlet on a second side for the release of an active ingredient, and a reservoir, which is arranged in a cavity of the housing and is designed to receive a liquid containing an active ingredient.

A second embodiment describes an implant according to embodiment 1, wherein the implant within the housing further comprises an anti-piercing means which, together with a wall of the housing, defines a channel, wherein the channel is designed to guide active ingredient from the reservoir to the outlet.

A third embodiment describes an implant according to the second embodiment, wherein the anti-piercing means comprises a surface structure which defines a distance of the anti-piercing means from the reservoir and/or from the housing. Such a distance can, for example, in each case be at least 0.5 mm or at least 1 mm.

A fourth embodiment describes an implant according to any of the preceding embodiments, wherein the reservoir comprises an open-pore material.

A fifth embodiment describes an implant according to any of the preceding embodiments, wherein the reservoir comprises a reticulated foam, sintered plastics particles, pressed plastics fibers, pressed natural fibers, or bonded plastics fibers.

A sixth embodiment describes an implant according to any of the preceding embodiments, wherein the reservoir comprises a crosslinked salt of polyacrylic acid, a crosslinked polyacrylamide, a salt of a crosslinked polystyrene sulfonic acid, a polysaccharide, or a polysaccharide derivative.

A seventh embodiment describes an implant according to any of the preceding embodiments, wherein the reservoir is arranged within the housing and is designed to allow the free circulation of a liquid containing an active ingredient between the septum and the outlet.

An eighth embodiment describes an implant according to any of the preceding embodiments, wherein the septum comprises a flexible, self-sealing polymer, wherein the polymer preferably comprises a polyhalogenated olefin, a silicone, or a thermoplastic elastomer.

A ninth embodiment describes an implant according to any of the preceding embodiments, wherein the septum comprises a convexly curved shape.

A tenth embodiment then describes an implant according to any of the preceding embodiments, wherein the septum is designed to expand elastically when a liquid is injected into the cavity of the housing.

An eleventh embodiment describes an implant according to any of the preceding embodiments, wherein the second side of the housing comprises a substantially planar shape.

A twelfth embodiment describes an implant according to any of the preceding embodiments, wherein the inner diameter of the outlet determines the delivery rate of an active ingredient from the reservoir.

A thirteenth embodiment describes an implant according to any of the preceding embodiments, which further comprises a fastening element designed to be connected to body tissue.

A fourteenth embodiment describes an implant according to any of the preceding embodiments, wherein the reservoir is filled with a liquid containing an active ingredient.

A fifteenth embodiment describes an implant according to any of the preceding embodiments which is designed, in a subcutaneously implanted state, to be filled with a liquid containing an active ingredient by injecting the liquid through the septum into the housing, preferably into the reservoir.

### DETAILED DESCRIPTION

With respect to the embodiments described herein, the elements of which “contain,” or “comprise,” a particular feature (for example, a material), in principle, a further embodiment is always contemplated in which the relevant element consists solely of the feature, i.e., does not comprise any other constituents. The word “comprise” or “comprising” is used herein synonymously with the word “contain” or “containing.”

In one embodiment, if an element is denoted by the singular, an embodiment is also contemplated in which more than one such element is present. The use of a term for an element in the plural in principle also includes an embodiment in which only a single corresponding element is included.

Unless otherwise indicated or clearly excluded from the context, it is possible in principle, and is hereby clearly contemplated, that features of different embodiments may also be present in the other embodiments described herein. Likewise, it is contemplated in principle that all features described herein in connection with a method are also applicable to the products and devices described herein, and vice versa. All such considered combinations are not explicitly listed in all instances, simply in order to keep the description brief. Technical solutions known to be equivalent to the features described herein are also intended in principle to be included by the scope of the invention.

A first aspect of the invention relates, in a first embodiment, to an implant for the sustained subcutaneous release of an active ingredient, which implant comprises a housing, which is delimited on a first side by a septum and comprises an outlet on a second side for the release of an active ingredient, and a reservoir, which is arranged in a cavity of the housing and is designed to receive a liquid containing an active ingredient.

The implant comprises a housing which is preferably made of a biocompatible material. The housing may comprise a plastics or a metal, or both. The housing can have a multipart design. The housing comprises a septum which is preferably formed from a soft, stretchable material. The septum is preferably designed to be pierced by means of a medical cannula in order to inject a liquid into the housing. The septum is attached to a first side of the housing. This first side is preferably designed to be aligned in the direction of the skin's surface when the implant is in the implanted state, for example a subcutaneous implantation, so that the septum is accessible from the outside by means of a cannula. The housing comprises a cavity in which a reservoir is arranged. The reservoir is designed to receive a liquid containing an active ingredient. This means that an aqueous or oily liquid can reach the interior of the reservoir. For this purpose, the reservoir can comprise an open porosity, as described in more detail below.

Furthermore, the implant comprises an outlet on a second side of the housing in order to deliver active ingredient from the housing to the outside. This can be done by diffusion, through the outlet, of a substance dissolved or suspended in a liquid containing an active ingredient, or by such a liquid flowing through the outlet out of the housing, or both.

The outlet can comprise a valve or a filter in order to prevent the penetration of high-molecular-weight substances, germs or other particles into the implant. For example, a filter having an exclusion volume of 220 nm, 200 nm, 100 nm, 75 nm or 50 nm can be attached at the outlet. The filter can comprise, for example, polyether sulfone (PES), polyvinylidene fluoride (PVDF), or polytetrafluoroethylene (PTFE). The valve or the filter can also define the release kinetics of the active ingredient.

The second side of the housing is arranged opposite the first side of the housing. When the implant is in an implanted state, the second side can be aligned with the inside of the patient's body, for example in the direction of a tissue to be treated, for example a joint. This allows targeted local delivery of an active ingredient to a target tissue.

The housing can be disk-shaped. The housing can, for example, comprise the shape of a flat cylinder. The housing can have a multipart design, wherein the different parts can be permanently connected to one another in a liquid-tight manner, for example by means of a click or snap closure or latch. With the exception of the outlets, the housing can be impermeable to liquid. The housing can comprise a metal or a plastic. The housing preferably comprises a biocompatible material.

In some embodiments, the implant further comprises an anti-piercing means within the housing. The anti-piercing means can be designed to prevent the housing from being completely pierced with a cannula on the first and the second side. The anti-piercing means can in particular be designed to prevent the housing from being pierced on the second side when a cannula is inserted through the septum on the first side of the housing.

This anti-piercing means, preferably together with a wall of the housing, defines a channel. For this purpose, the anti-piercing means can be arranged, for example, at a distance from the wall of the housing, so that a liquid-permeable interstice is formed. Such an interstice defines a channel which is designed to guide a liquid containing an active ingredient from the reservoir to the outlet. For example, an active ingredient dissolved or suspended in a liquid can diffuse from the reservoir through the channel to the outlet.

The anti-piercing means can comprise a metal, a plastics material, a ceramic material, or a plastics-metal composite. In one embodiment, the anti-piercing means comprises a material which, due to its strength, cannot be pierced by a medical injection cannula according to ISO 7864:2016.

The anti-piercing means can comprise a surface structure. This surface structure can define a distance between the anti-piercing means and the reservoir. Alternatively or additionally, the surface structure can define a distance between the anti-piercing means and the housing, in particular from the second side of the housing. This distance can be designed to allow the diffusion of a dissolved active ingredient around the anti-piercing means, for example from the reservoir to the outlet.

The surface structure can, for example, be a curvature of a portion of the surface of the anti-piercing means. The surface structure can result in the anti-piercing means not resting completely flat against the housing, but instead comprising self-supporting regions at a plurality of points which form interstices between the anti-piercing means and the housing. In a similar manner, the surface structure can result in the reservoir not resting completely flat against the surface of the anti-piercing means.

The reservoir can comprise an open-pore material. As a result, a liquid containing an active ingredient can pass into the interior of the reservoir, and can be accommodated in the reservoir.

The reservoir can comprise, for example, a reticulated foam, sintered plastics particles, pressed plastics fibers, pressed natural fibers, or bonded plastics fibers. The foam can, for example, comprise polyethylene, polypropylene, polyamide, polyacrylic or polyurethane. Suitable medical foams are available inter alia from Porex, Reinbek (Germany). Examples of foams are described in EP2480408A1 and EP1581268A1, which are hereby incorporated fully by reference. The foam can comprise or consist of a fiber-containing or fiber-free material.

The reservoir can comprise a hydrogel. Hydrogels usually contain crosslinked polymers which form a sponge-like structure. The reservoir can comprise, for example, a crosslinked salt of polyacrylic acid, a crosslinked polyacrylamide, a salt of a crosslinked polystyrene sulfonic acid, a polysaccharide, or a polysaccharide derivative. Further examples include acrylamide copolymers, for example a copolymer of acrylamide and acrylic acid, or a copolymer of acrylamide and sodium acrylate. Further examples of substances that can be used for the reservoir are commercially available superabsorbents, such as HySorb® and SAVIVA® (BASF, Ludwigshafen, Germany). Examples of polysaccharides include cellulose, guar gum, sodium alginate, chitosan, carrageenan, cyclodextrin and amylose. Examples of polysaccharide derivatives include carboxymethyl cellulose and carboxymethylamylose.

In some embodiments, the reservoir is arranged within the housing and designed to allow free circulation of a liquid containing an active ingredient between the septum and the outlet. This can be achieved, for example, by a movable arrangement of the reservoir within the housing. For example, the reservoir can be freely movable relative to the housing, the septum, and/or the anti-piercing means. In some embodiments, the reservoir is not fixedly connected to the housing, the septum, and/or the anti-piercing means. In this way, free diffusion of a dissolved active ingredient within the housing, as well as in and also out of the reservoir, can be achieved.

In some embodiments, the septum comprises a flexible, self-sealing polymer. The polymer can comprise a polyhalogenated olefin, a silicone, or a thermoplastic elastomer. The septum is preferably designed to close the housing, with the exception of the outlets of the housing, in a liquid-tight manner after the septum has been pierced with a cannula. The septum is preferably stretchable and exhibits rubber-elastic properties, as is known, for example, from septa in chemical bottles. As a result and as described above, the septum can, on the one hand, heal itself after having been pierced and expand into the housing upon injection of a liquid in order to provide the indexed liquid with a larger volume within the housing, i.e., the cavity in the housing can be enlarged by the expansion of the septum. The septum can thus be designed to expand elastically when a liquid is injected into the cavity of the housing.

The septum preferably comprises a biocompatible material.

The septum can, for example, comprise a substantially planar or convexly curved shape. The septum can comprise a circular shape.

In one embodiment, the septum comprises a material which can be pierced with a medical injection cannula according to ISO 7864:2016 with a force of less than 100 N. The septum is preferably connected to the housing in a liquid-tight manner.

In one embodiment, the second side of the housing comprises a substantially flat shape, for example a disk shape. On the outside, the second side of the housing can comprise an outwardly curved structure which can serve as a spacer. These structures can, for example, comprise the shape of outwardly protruding ridges or nubs.

The implant according to the invention can be adapted for faster or slower release kinetics of active ingredients. For this purpose, for example, the inner diameter and/or the number of outlets can be reduced or increased. Accordingly, the inner diameter of the outlet can determine the delivery speed of an active ingredient from the reservoir. The diameter of an outlet can be, for example, at most 0.5 mm or at most 0.25 mm.

The implant described herein can furthermore comprise a fastening element which is designed to be connected to body tissue. For example, an eyelet can be arranged on the outside of the housing, which eyelet can be sewn to a target fabric. A plurality of such elements can also be provided which, for example, can be arranged circumferentially on the outside of the housing. This can better ensure that a targeted local release of an active ingredient can take place.

In some embodiments, the reservoir can be filled with a liquid containing an active ingredient. For example, within the scope of the manufacturing process, the reservoir can already be pre-filled with a liquid containing an active ingredient. This can better ensure that the liquid containing an active ingredient remains sterile until application, and that an exactly predetermined amount of an active ingredient is contained in the implant. In addition, in this way, an equilibrium of the active ingredient concentration can be better adjusted between the reservoir and the other cavity within the housing. The reservoir can be filled as described herein by injection through the septum. Alternatively, a pre-filled reservoir can be installed in the housing so that the septum does not need to be breached during the manufacturing process.

In principle, the reservoir can be filled with any active ingredient that can be dissolved or suspended in a liquid. Anti-rheumatic active ingredients are particularly preferred. For example, a series of active ingredient groups is available for the treatment of rheumatoid arthritis and psoriatic arthritis. These active ingredient groups include analgesics, non-steroidal antiphlogistics, glucocorticoids and basic therapeutics (disease modifying anti-rheumatic drugs, DMARD). In addition, there are therapeutic antibodies (biologicals) which inhibit the signal paths of α-TNF (α-tumor necrosis factor) or interleukin IL-17. Also usable are low-molecular-weight active ingredients which suppress the immune system. Such immunomodulators comprise, for example, cyclosporine A (CAS 59865-13-3), tacrolimus (CAS 104987-11-3) and everolimus (CAS 159351-69-6). Further examples of active ingredients are cortisone (CAS 53-06-5), betamethasone (CAS 378-44-9), triamcinolone (CAS 124-94-7), dexamethasone (CAS 50-02-2), dexamethasone phosphate (CAS 2392-39-4) and apremilast (CAS 608141-41-9).

The mechanism of action of cyclosporine A can be attributed to the formation of a complex of cyclosporine A and immunophilin cyclophilin. Said complex inhibits the phosphatase calcineurin. The enzyme calcineurin is in turn essential for the formation of α-TNF, wherein α-TNF is a key compound at the beginning of the inflammatory cascades. By inhibiting the synthesis of α-TNF, the following inflammatory cascades can accordingly also be inhibited.

In systemic drug therapy, serum active ingredient concentrations in the range of about 75 μg/l to 175 μg/l for cyclosporine A and of 5 μg/l to 20 μg/l for tacrolimus can be achieved. Higher systemic active ingredient concentrations are not systemically possible for longer lasting treatment due to the risk of undesired toxic side effects for the rest of the organism. This means that in systemic therapy, the active ingredient concentrations in the tissue of the joint capsule (Membrana fibrosa and Membrana synovialis) are very low and can thus have only a limited immunomodulating effect on the inflammatory process. By contrast, with the aid of the implant according to the invention, active ingredients can be applied subcutaneously to the tissue of the joint capsule directly or in the vicinity of the joint capsule in order to reduce the chronic inflammation processes in the joint region of patients with rheumatoid arthritis or psoriatic arthritis in order to achieve a sufficiently high concentration of these active ingredients locally.

The implant can also be filled with active ingredient after implantation in the patient. If the implant is implanted subcutaneously, for example, the reservoir can be filled from the outside by injection through the septum, without the implant having to be exposed or explanted. A further embodiment accordingly describes an implant which is designed to be filled with a liquid containing an active ingredient in a subcutaneously implanted state by injecting the liquid into the housing through the septum. The liquid can thereby be injected into the cavity of the housing, or preferably directly into the reservoir arranged in the cavity of the housing.

A further aspect of the invention relates to a method for filling an implant, wherein the implant comprises a reservoir and a septum, wherein, when the implant is in an implanted state, the septum is pierced from the outside with a cannula in order to introduce a liquid containing an active ingredient into the reservoir. In this case, the liquid can be injected directly into the reservoir, or can be introduced into the cavity outside the reservoir, and then be received by the reservoir. After the injection process, the restoring force of the elastic material of the septum allows the septum to seal itself again to be liquid tight. In one embodiment, the implant is filled in a non-implanted state, i.e., in vitro.

A further aspect relates to a medical treatment method, wherein an active ingredient is delivered with a delay to a joint by means of an implant described herein. For this purpose, the implant can, for example, be implanted subcutaneously in the region of a joint and optionally connected to soft tissue, for example using the fastening elements described herein.

A further aspect of the invention relates to an anti-rheumatic active ingredient for use in a therapeutic procedure. The active ingredient can thereby be administered using the implants and methods described herein. The active ingredient can thereby be delivered from a reservoir in a sustained manner via an outlet of the implant, as described herein.

### EXAMPLES

The invention is further illustrated below using examples which are, however, not to be understood as limiting. It will be apparent to a person skilled in the art that other equivalent means may be used similarly in place of the features described here.

### FIGURES

FIG. 1 shows by way of example a cross-sectional view of a first embodiment of an implant 100 according to the invention. The implant comprises a disk-shaped housing 101 which is designed in two parts in the example shown here. On a first side 111, a septum 102 is introduced into the upper part of the housing in a liquid-tight manner. In the lower part of the housing 101, outlets 103 are incorporated at a second side of the housing 112, which outlets connect the cavity 105 in the interior of the housing to the external environment of the housing in a liquid-conducting manner. Furthermore, a reservoir 104 is arranged in the cavity 105 of the housing 101 and designed to receive a liquid containing an active ingredient. An anti-piercing means 106 is arranged between the reservoir 104 and the second side of the housing 112, which anti-piercing means 106 can prevent piercing by an injection cannula. The reservoir comprises a material with open porosity which allows liquid to penetrate and be stored. The anti-piercing means can be formed, for example, from a solid metal, such as, for example, medical stainless steel or titanium. The anti- piercing means 106 is at a distance from a side wall 107 of the housing, in order to form a channel 108 therebetween. The channel 108 allows active ingredients to be transported from the first side 111 to the second side 112 of the cavity 105 so that active ingredients can be delivered through the outlets 103 to the outside.

FIG. 2 shows a further embodiment of an implant 100 according to the invention in an isometric plan view. The implant comprises a septum 102 on the upper side. The housing 101 comprises a plurality of fastening elements 113 circumferentially about the lateral edge which are embodied here as holes in a laterally protruding part of the housing 101. The fastening elements 113 allow the implant to be fixed to a target tissue, for example allow the implant to be stitched to soft tissue in the region of a joint.

FIG. 3 shows a cross-sectional view of the filling of an implant 100 according to the invention with the aid of a cannula 200. Here, the septum 102 is pierced with the aid of a cannula 200, and a liquid containing an active ingredient (shown symbolically as drops in the illustration) is introduced into the housing. The liquid can simultaneously or subsequently be received in the reservoir 104. The anti-piercing means 106 prevents the second side 112 of the housing 101 from being pierced by the cannula 200. The subjacent tissue is thereby protected when the implant is filled when in the implanted state.

FIG. 4 shows a cross-sectional view of the delivery of a liquid containing an active ingredient from an implant 100 according to the invention. The liquid and/or an active ingredient dissolved therein is delivered from the reservoir 104 to the cavity of the housing 101 and passes through the outlets 103 on the second side 112 of the housing 101 to the outside. The anti-piercing means 106 is arranged such that it allows the active ingredient to be transported from the reservoir 104 to the outlets 103.

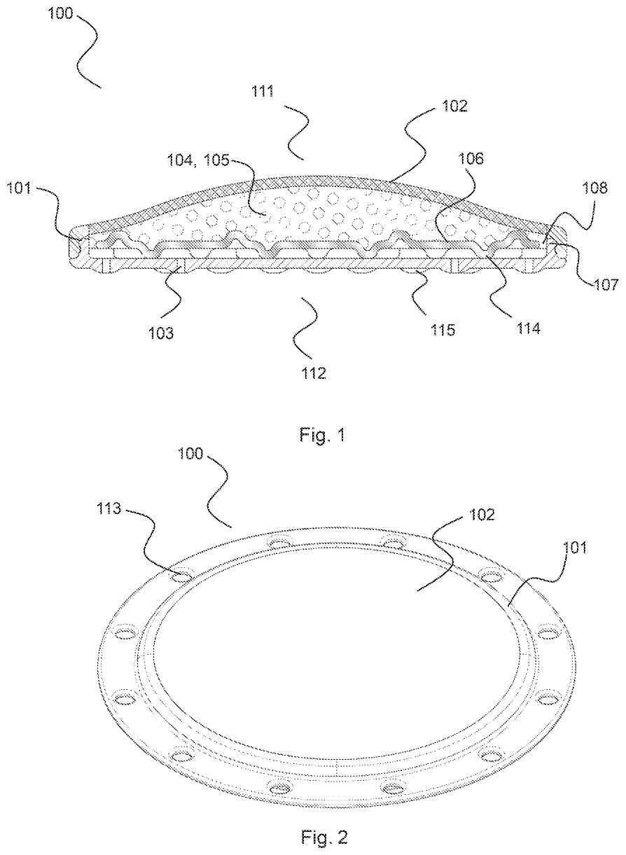

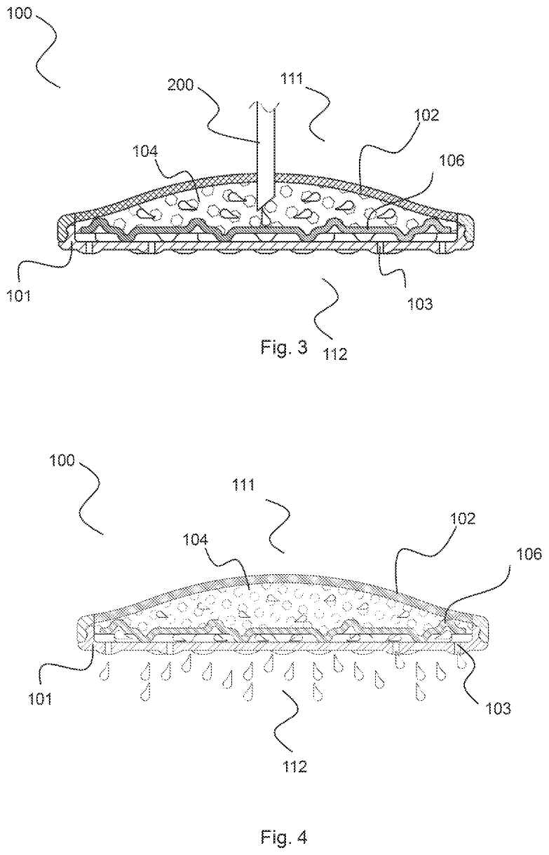

### LIST OF REFERENCE NUMERALS

100 Implant
101 Housing
102 Septum
103 Outlet
104 Reservoir
105 Cavity
106 Anti-piercing means
107 Wall
108 Channel
111 First side of the housing
112 Second side of the housing
113 Fastening element
114 Surface structure
200 Cannula

1. An implant for the sustained subcutaneous release of an active ingredient, comprising a housing, which is delimited on a first side by a septum and comprises an outlet on a second side for the release of an active ingredient, and a reservoir, which is arranged in a cavity of the housing and is designed to receive a liquid containing an active ingredient.

2. The implant according to claim 1, wherein the implant further comprises an anti-piercing means within the housing which, together with a wall of the housing, defines a channel, wherein the channel is designed to guide active ingredient from the reservoir to the outlet.

3. The implant according to claim 2, wherein the anti-piercing means comprises a surface structure which defines a distance of the anti-piercing means from the reservoir and/or from the housing.

4. The implant according to claim 1, wherein the reservoir comprises an open-pore material.

5. The implant according to claim 1, wherein the reservoir comprises reticulated foam, sintered plastics particles, pressed plastics fibers, pressed natural fibers, or bonded plastics fibers.

6. The implant according to claim 1, wherein the reservoir comprises a crosslinked salt of polyacrylic acid, a crosslinked polyacrylamide, a salt of a crosslinked polystyrene sulfonic acid, a polysaccharide, or a polysaccharide derivative.

7. The implant according to claim 1, wherein the reservoir is arranged within the housing and is designed to allow free circulation of a liquid containing an active ingredient between the septum and the outlet.

8. The implant according to claim 1, wherein the septum comprises a flexible, self-sealing polymer, wherein the polymer preferably comprises a polyhalogenated olefin, a silicone, or a thermoplastic elastomer.

9. The implant according to claim 1, wherein the septum comprises a convexly curved shape.

10. The implant according to claim 1, wherein the septum is designed to expand resiliently when a liquid is injected into the cavity of the housing.

11. The implant according to claim 1, wherein the second side of the housing comprises a substantially planar shape.

12. The implant according to claim 1, wherein the inner diameter of the outlet determines the delivery rate of an active ingredient from the reservoir.

13. The implant according to claim 1, which further comprises a fastening element designed to be connected to body tissue.

14. The implant according to claim 1, wherein the reservoir is filled with a liquid containing an active ingredient.

15. The implant according to claim 1, which is designed, in a subcutaneously implanted state, to be filled with a liquid containing an active ingredient by injecting the liquid through the septum into the housing, preferably into the reservoir.
